(19) United States
(12) Patent Application Publication
(10) Pub. No.: US 2012/0076260 A1
(43) Pub. Date: Mar. 29, 2012

(54) Image processing apparatus and method, computer executable program, and radiation imaging system

(75) Inventors: Yusuke Kitagawa, Kanagawa (JP); Noriaki Ida, Kanagawa (JP)

(73) Assignee: FUJIFILM Corporation, Tokyo (JP)

(21) Appl. No.: 13/137,882

(22) Filed: Sep. 20, 2011

(30) Foreign Application Priority Data
Sep. 28, 2010 (JP) 2010-216752

(51) Int. Cl. A61B 6/02; G06K 9/00

(52) U.S. Cl. 378/41; 382/128

(57) ABSTRACT

In three dimensional X-ray imaging, a user interface is for search support with plural images, which are obtained by imaging an object upon applying X-rays thereto from an X-ray source. There is a display region for selecting two parallax images of one image pair among the plural images. Right and left buttons are for determining a direction to shift a view center of an image pair by selection from first and second directions in which an irradiation angle changes. A moving area image and line segment are for setting a small search area limited within an initial search area by considering the determined first or second direction, a first image pair specified previously, and a current second image pair in multi-stage binary search. Two parallax images of a third image pair are acquired from the small search area, to raise efficiency in search from the initial search area.

### BACKGROUND OF THE INVENTION

1. Field of the Invention

The present invention relates to an image processing apparatus and method, computer executable program, and radiation imaging system. More particularly, the present invention relates to an image processing apparatus and method in which search of images can be quickened by efficient procedure, computer executable program, and radiation imaging system.

2. Description Related to the Prior Art

JP-A 2010-131170 discloses tomosynthesis imaging, in which X-rays are applied to an object at various angles by moving an X-ray source as radiation source. Images of the objects are formed, and added up to create a tomographic image in which surfaces of tomography are effectively enhanced.

JP-A 2010-131170 discloses stereoscopy of two images (parallax images) having parallax among a plurality of image obtained by tomosynthesis imaging. A region of interest (ROI) is specified by the stereoscopy so as to obtain tomographic image of the region of interest.

In the field of medical imaging with X-rays, two X-ray sources are disposed to form a pair of parallax images by radiation imaging. Stereoscopic diagnosis is possible according to two X-ray images of a stereo image pair. See U.S. Pat. No. 5,090,038 (corresponding to JP-A 3-123537).

U.S. Pat. No. 5,090,038 discloses radiation imaging in which an X-ray source and an image intensifier (II) are rotated about an object. X-rays are applied to the object intermittently, to form plural X-ray images at a constant frame rate. It is possible to adjust the depth of stereoscopy because a depth setting unit is used for setting a frame rate (parallax angle) of parallax images for the stereoscopy to increase or decrease the parallax angle.

In general, parallax images are viewed stereoscopically in JP-A 2010-131170 as tomosynthesis imaging, because a region of interest should be easily recognized in the images. However, the number of the images according to the tomosynthesis imaging is as high as 40-80. It is likely that a region of interest is extremely difficult to recognize according to selection of parallax images. Smooth and quick diagnosis cannot be conducted easily, as tomographic images according to a stereo image pair of parallax images with the region of interest are formed only with very long time.

U.S. Pat. No. 5,090,038 discloses the depth setting unit for setting the frame rate. However, viewing the images for selection by changing their parallax angle require considerably great labor and long time. Should the depth setting unit be combined with the construction of JP-A 2010-131170, it is impossible to shorten time for the selection of the parallax image very effectively.

### SUMMARY OF THE INVENTION

In view of the foregoing problems, an object of the present invention is to provide an image processing apparatus and method in which search of images can be quickened by efficient procedure, computer executable program, and radiation imaging system.

In order to achieve the above and other objects and advantages of this invention, an image processing apparatus for plural images is provided, the plural images being obtained by imaging of an object upon application of radiation at plural irradiation angles from irradiation positions within an area between first and second irradiation positions. A display control unit displays two of the images on a display panel stereoscopically with a predetermined parallax angle, the two images being formed with the radiation from irradiation positions which are symmetric with one another with respect to a view center determined specifically within the area between said first and second irradiation positions. A first input unit selects a shift direction of the view center from forward and backward directions defined between the first and second irradiation positions. A search processing unit checks whether an earlier view center specified previously is located on a specified side extending in the shift direction from the view center being current, and if the earlier view center is located on the specified side, determines a search area between the current view center and a selected one of earlier view centers which is nearest to the current view center, and if the earlier view center is located opposite to the specified side, determines a search area between the current view center and a position which is located on the specified side and farthest from the current view center. An acquisition device is adapted to acquiring two new images from the search area for display on the display panel by determining a new view center thereof at a center of the search area.

Initially before operating the first input unit, the view center is a center between the first and second irradiation positions.

Furthermore, a second input unit changes the parallax angle.

Furthermore, a second input unit shifts the view center to change over the two images on the display panel to two other images.

Furthermore, a second input unit changes the parallax angle with reference to a first image of the two images in order to change over a second image of the two images displayed on the display panel to another image.

Furthermore, an image processing device creates a tomographic image in which a region of interest of the object is enhanced by adding up data of the plural images according to the two images of the third image pair.

The tomographic image is a tomosynthesis image.

The image processing apparatus comprises a computer apparatus for viewing the images and editing a medical report.

The image processing apparatus comprises a computer apparatus in connection with a portable type of radiation imaging system.

Also, an image processing method for plural images is provided, the plural images being obtained by changing an irradiation angle of a radiation source to change an irradiation position thereof, and by imaging an object upon applying radiation thereto from the radiation source. In the image processing method, two images of one image pair are selected among the plural images. A direction to shift a view center of an image pair is determined by selection from first and second directions in which the irradiation angle changes relative to a reference angle. If the first direction is determined with the determining step and if a view center of a first image pair specified previously is disposed in the first direction, a small search area within an initial search area is set from a view center of a current second image pair to the view center of the first image pair, and if the first direction is determined with the determining step and if the view center of the first image pair is disposed in the second direction, a small search area is set from the view center of the second image pair to an end point of the irradiation position disposed in the first direction. Two images of a third image pair are acquired from the small search area.

Also, a computer executable program for search on plural images is provided, the plural images being obtained by changing an irradiation angle of a radiation source to change an irradiation position thereof, and by imaging an object upon applying radiation thereto from the radiation source. There is a selecting program code for selecting two images of one image pair among the plural images. A determining program code is for determining a direction to shift a view center of an image pair by selection from first and second directions in which the irradiation angle changes relative to a reference angle. A setting program code is for, if the first direction is determined with the determining program code and if a view center of a first image pair specified previously is disposed in the first direction, setting a small search area within an initial search area from a view center of a current second image pair to the view center of the first image pair, and for, if the first direction is determined with the determining program code and if the view center of the first image pair is disposed in the second direction, setting a small search area from the view center of the second image pair to an end point of the irradiation position disposed in the first direction. An acquiring program code is for acquiring two images of a third image pair from the small search area.

Also, a radiation imaging system is provided, and includes a radiation source for applying radiation to an object. A detection device detects an image by receiving the radiation transmitted by the object. A moving device moves the radiation source relative to the detection device to change an irradiation angle of the radiation source relative to the object so as to apply the radiation to the object in plural irradiation positions. The radiation imaging system includes an image processing apparatus as defined above, for processing the image.

The moving device arcuately moves the radiation source.

Also, a user interface for search on plural images is provided, the plural images being obtained by changing an irradiation angle of a radiation source to change an irradiation position thereof, and by imaging an object upon applying radiation thereto from the radiation source. There is a selecting region for selecting two images of one image pair among the plural images. A determining region is for determining a direction to shift a view center of an image pair by selection from first and second directions in which the irradiation angle changes relative to a reference angle. A setting region is for, if the first direction is determined with the determining region and if a view center of a first image pair specified previously is disposed in the first direction, setting a small search area within an initial search area from a view center of a current second image pair to the view center of the first image pair, and for, if the first direction is determined with the determining region and if the view center of the first image pair is disposed in the second direction, setting a small search area from the view center of the second image pair to an end point of the irradiation position disposed in the first direction. An acquiring region is for acquiring two images of a third image pair from the small search area.

Consequently, search of images can be quickened by efficient procedure, because the search is conducted in two or more stages in a binary search.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating a radiation imaging system;

FIG. 2 is a block diagram illustrating an imaging controller;

FIG. 3 is a chart illustrating tomosynthesis imaging;

FIG. 4 is a block diagram illustrating a viewer terminal apparatus;

FIG. 5 is a block diagram illustrating circuit elements in the viewer terminal apparatus;

FIG. 6 is an explanatory view illustrating assignment of identifiers to image data;

FIG. 7 is a plan illustrating an image search window;

FIG. 8 is a table illustrating statuses of operation buttons and a small search area for parallax images;

FIG. 9A is an explanatory view illustrating a default state of a status diagram;

FIG. 9B is an explanatory view illustrating a state of the status diagram after a first search stage;

FIG. 9C is an explanatory view illustrating a state of the status diagram after a second search stage;

FIG. 9D is an explanatory view illustrating a state of the status diagram after a third search stage.

### DETAILED DESCRIPTION OF THE PREFERRED EMBODIMENT(S) OF THE PRESENT INVENTION

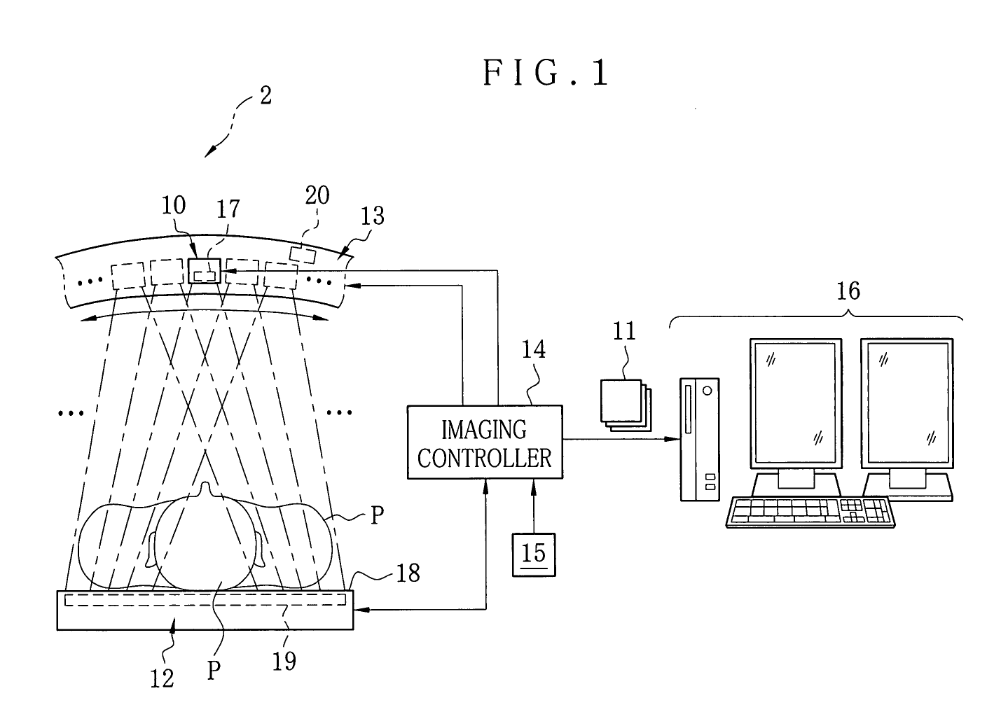

In FIG. 1, an X-ray imaging system 2 as radiation imaging system includes an X-ray source 10, an imaging cassette 12, a moving device 13, and an imaging controller 14. The imaging cassette 12 detects a component of X-rays transmitted through an object P after emission from the X-ray source 10. Information of an X-ray image 11 or image data is output by the imaging cassette 12. The moving device 13 moves the X-ray source 10 in an arrow direction. The imaging controller 14 controls the X-ray source 10, the imaging cassette 12 and the moving device 13 for suitable imaging. Each of the X-ray source 10, the imaging cassette 12 and the moving device 13 is connected to the imaging controller 14 by a cable in a wired manner, and is supplied with power by the imaging controller 14.

An input interface 15 is an input device for inputting imaging conditions, for example, a body part, voltage, current, radiation time for an X-ray tube 17 in the X-ray source 10, and the like. The imaging controller 14 controls the X-ray source 10 and the imaging cassette 12 for synchronism in the operation according to the imaging conditions. When the imaging controller 14 receives an instruction signal for radiation from the input interface 15, the imaging controller 14 controls the X-ray source 10 and the imaging cassette 12 in synchronism by sending a signal to the imaging cassette 12.

A viewer terminal apparatus 16 or image processing apparatus or image search apparatus is connected to the imaging controller 14. The X-ray image 11 generated from the imaging cassette 12 is input by the imaging controller 14 to the viewer terminal apparatus 16. Examples of the viewer terminal apparatus 16 are a personal computer, workstation and the like. The viewer terminal apparatus 16 processes the X-ray image 11 for image processing of various functions, and supports display of the X-ray image 11 and editing of a radiology report as a result of viewing the X-ray image 11.

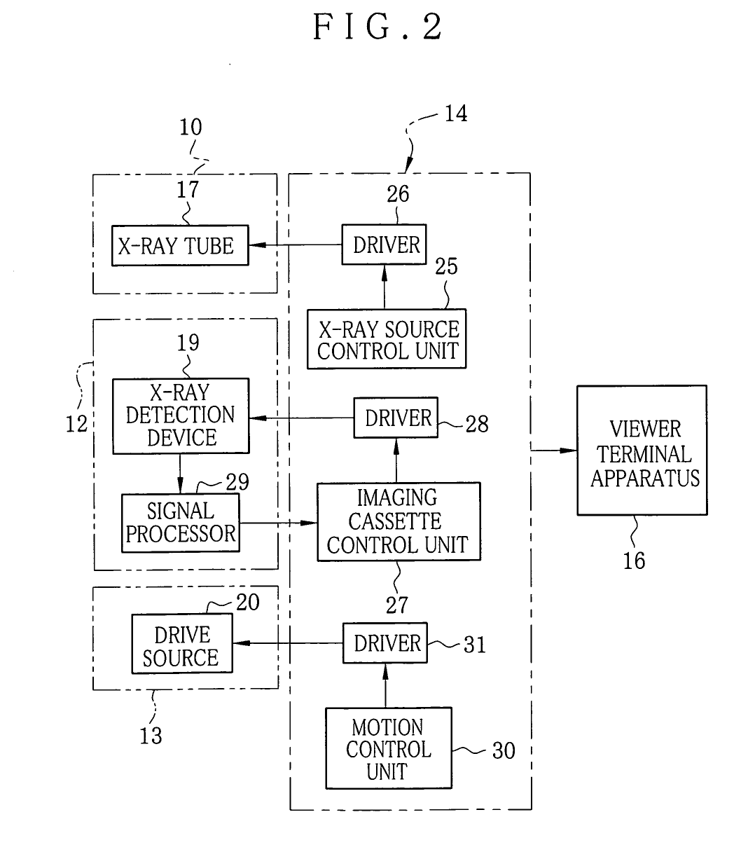

The X-ray source 10 includes the X-ray tube 17, a collimator (not shown) and the like. In FIG. 2, a high voltage source 26 or driver applies high voltage to the X-ray tube 17. The collimator limits a field of X-rays generated by the X-ray tube 17.

The imaging cassette 12 is in a quadrilateral shape. A receiving surface 18 or sensor surface of the imaging cassette 12 is directed to the X-ray source 10 as depicted in the drawing. The imaging cassette 12 is disposed under the object P, or can be positioned at a shoulder, knee or the like of the body suitably.

An X-ray detection device 19 or FPD device or flat panel detection device is incorporated in the imaging cassette 12. The X-ray detection device 19 is a device including a matrix board in which plural pixels are arranged in a two-dimensional manner inclusive of thin film transistors (TFT) and X-ray detection elements. When the thin film transistors are turned off, the X-ray detection device 19 stores charge in the X-ray detection elements according to an amount of the X-rays. The thin film transistors are turned on for externally reading the charge stored in the X-ray detection elements. A signal processor 29 of FIG. 2 has an integration amplifier, which converts the charge into a voltage signal, which is converted digitally by an A/D converter in the signal processor 29. Thus, a digital form of the X-ray image 11 is created.

A hook (not shown) is disposed to suspend the X-ray source 10. A support shaft or moving shaft is disposed to support the X-ray source 10 by use of the hook. A rail groove is formed in the support shaft and extends in its longitudinal direction. The hook is movably secured to the rail groove. The hook moves along the rail groove in the arrow direction. The moving device 13 is constituted by the hook and the rail groove. A drive source 20 or motor is incorporated in the moving device 13, includes a stepping motor and the like, and actuated by the imaging controller 14. The hook and the X-ray source 10 are moved along the rail groove when the drive source 20 is actuated. When the drive source 20 stops, the X-ray source 10 stops in a desired position of the support shaft (irradiation position). The imaging controller 14 counts the voltage pulses generated by the drive source 20 for driving the stepping motor, and detects a position of the X-ray source 10 on the support shaft according to the counted number.

The imaging controller 14 controls the drive source 20, and moves the X-ray source 10 to one of plural positions (for example, 40-80 positions) predetermined for the support shaft or moving shaft according to the imaging condition. Specifically, the X-ray source 10 is moved in a direction from the left end toward the right end in FIG. 1. At each time that the X-ray source 10 reaches one of the positions, the X-ray source 10 emits X-rays to the object P, so that the imaging cassette 12 detects X-rays. A plurality of X-ray images 11 are output by the imaging cassette 12 upon detection of X-rays applied at plural angles with changes in the position of the X-ray source 10.

In the embodiment, the X-ray source 10 is moved along the rail groove with the support shaft arcuately. However, a support shaft can be straight and used for moving the X-ray source 10 on a straight path instead of the curved path. In combination with the straight path, a pivotally moving mechanism can be associated with the hook to direct the X-ray source 10 to the imaging cassette 12. According to the curved path of the above embodiment, the X-ray source 10 is directed to the imaging cassette 12 simply without further mechanism. No pivotally moving mechanism is required. Also, a plurality of X-ray sources 10 can be used.

In FIG. 2, an X-ray source control unit 25 is incorporated in the imaging controller 14, and controls various elements in the X-ray source 10. The X-ray source control unit 25 causes the high voltage source 26 to control the X-ray tube 17, which emits X-rays in a determined condition and sequence.

An imaging cassette control unit 27 controls elements of the imaging cassette 12. A driver 28 is caused by the imaging cassette control unit 27 to control detection of the X-ray detection device 19 in the imaging cassette 12 for a predetermined sequence of operation. Also, the imaging cassette control unit 27 receives the X-ray image 11 from the signal processor 29 which includes an integration amplifier and an A/D converter, and transmits the X-ray image 11 to the viewer terminal apparatus 16.

A driver 31 drives the drive source 20 for the moving device 13. A motion control unit 30 causes the driver 31 to control the drive source 20. Thus, the X-ray source control unit 25, the imaging cassette control unit 27 and the motion control unit 30 cooperate together to form a plurality of X-ray images 11 by changing the position of the X-ray source 10 and applying X-rays at the various angles of the X-ray source 10. Tomosynthesis imaging is carried out to create a tomographic image or reconstructed image according to the plural X-ray images 11.

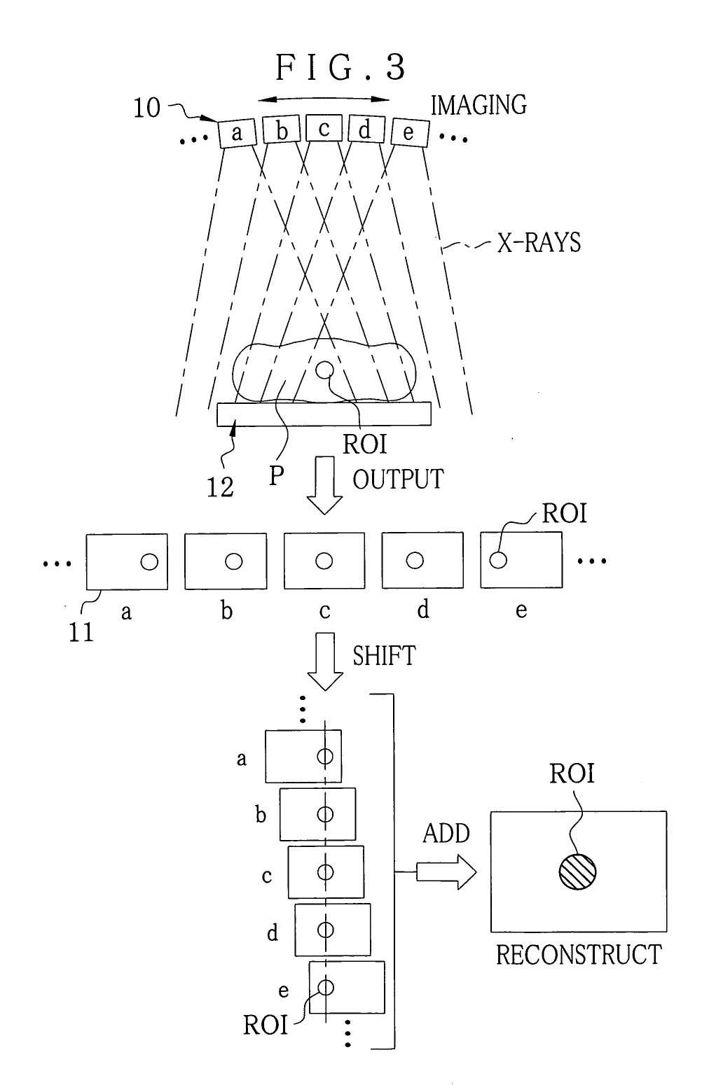

In FIG. 3, the viewer terminal apparatus 16 is schematically illustrated. The viewer terminal apparatus 16 changes the position of the X-ray source 10 for plural states to emit X-rays at various angles. According to the X-ray image 11 output by the imaging cassette 12, the viewer terminal apparatus 16 creates a tomographic image of the object P, specifically a tomographic image parallel to the receiving surface 18 of the imaging cassette 12 in a region of interest ROI of the object P. In a method of producing a tomographic image, the X-ray images 11 created by imaging in positions a, b, c, d and e are processed for shift processing in which positions of the ROI of the X-ray images 11 are registered with one another. Then the X-ray images 11 after the shift processing are added up, to create a reconstructed image in which the ROI is enhanced.

Other available examples of methods of creating tomographic images include a simple back projection method (tomographic back projection method) and a filtered back projection method. The simple back projection method is a method of back projection of plural images without use of the reconstruction filter, and obtaining a reconstructed image by addition processing after the back projection. One example of the filtered back projection method is a method of filtering plural images with a reconstruction filter as a convolution filter, back projection after the filtering, and obtaining a reconstructed image by addition processing. A second example of the filtered back projection method is a method of converting plural images according to the Fourier transform, replacing the images with data of frequency space, filtering the plural images with a reconstruction filter, back projection after the filtering, and obtaining a reconstructed image by addition processing.

The viewer terminal apparatus 16 is based on a computer, and has programs installed therein. Examples of the programs include an operation system (OS) as a control program, and an application program as a client program.

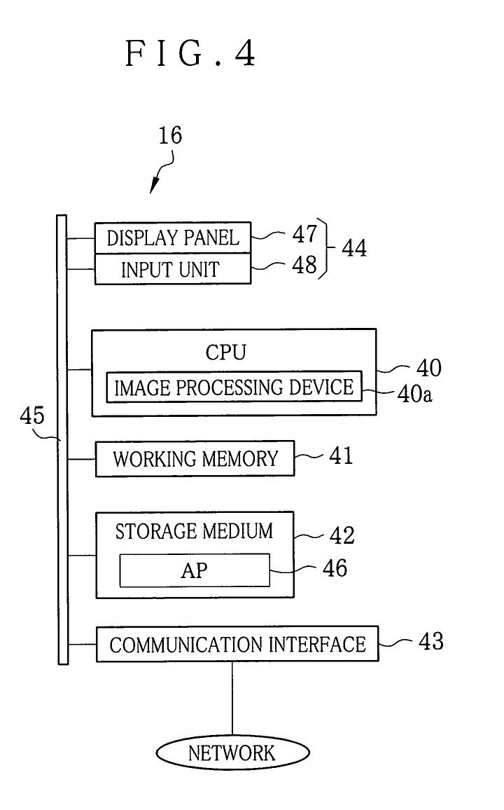

In FIG. 4, the computer constituting the viewer terminal apparatus 16 includes a CPU 40 (image processing device 40a), a working memory 41, a storage medium 42, a communication interface 43, and a console unit 44. A data bus 45 connects those elements with one another.

An example of the storage medium 42 is an HDD or hard disk drive. An application program 46 (AP) or computer executable program is stored in the storage medium 42 together with the control program.

The working memory 41 is connected in order to perform tasks in the CPU 40. The CPU 40 reads a control program stored in the storage medium 42, and loads the working memory 41 with the control program, and then controls various elements in the computer by processing according to the control program.

The communication interface 43 is a network interface for communication with the imaging controller 14. The console unit 44 includes display panels 47 and an input unit 48 (input means), for example, keyboard, mouse and the like.

The application program 46 is installed in the viewer terminal apparatus 16, and is a client program for radiology report editing as a support of radiological reading of images. The viewer terminal apparatus 16 operates according to the client program for display processing of the X-ray image 11 and editing processing of a radiology report.

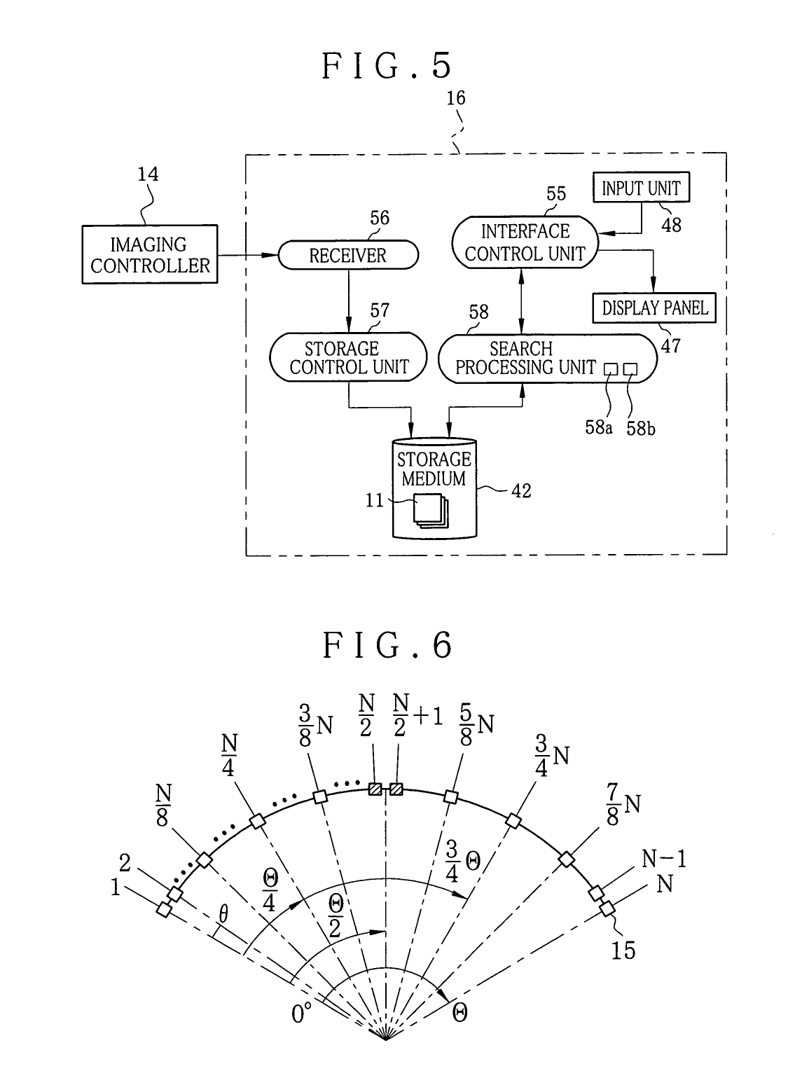

In FIG. 5, the CPU 40 includes an interface control unit 55 as display control unit (display control means) for the console unit, a receiver 56, a storage control unit 57 or writer, and a search processing unit 58 (search processing means). When the application program 46 is started up, those elements in the viewer terminal apparatus 16 are ready to function.

The viewer terminal apparatus 16 is constructed to include a terminal main unit with the CPU 40, and two display panels 47 connected to the main unit. See FIG. 1. A first one of the display panels 47 displays a display window for images. A second one of the display panels 47 displays an editing window for creating a radiology report.

The display window and the editing window are input interfaces according to the GUI (graphical user interface). The interface control unit 55 reads graphic data from the storage medium 42 according to inputs from the input unit 48, and outputs image data of window forms to the display panels 47 according to the graphic data. The interface control unit 55 receives inputs from the input unit 48 by use of the image data of window forms.

The display window and the editing window are caused to appear by startup in synchronism with one another. When a search query for an image to be read is input through the editing window, the interface control unit 55 causes the search processing unit 58 to retrieve the X-ray image 11 from the storage medium 42 according to the search query. The display window appears by control of the interface control unit 55 for the purpose of outputting the X-ray image 11 to the display panel 47 after being retrieved.

The display window displays the reconstructed image described above, and also various images such as 3D image, which is created according to two of the X-ray images 11 among the plural X-ray images 11 obtained by tomosynthesis imaging. A number of partial areas are disposed in the display window, including operation buttons, list boxes, input fields, icons and the like as tools in the GUI. A user can enter inputs by use of those tools in the input unit 48.

The receiver 56 receives the X-ray image 11 from the imaging controller 14, and outputs a command signal to the storage control unit 57 for storage. The storage control unit 57 in response writes the X-ray image 11 to the storage medium 42 after reception in the receiver 56. Also, the storage control unit 57 writes data of a radiology report created by use of the editing window.

The storage control unit 57 operates according to the counted number of voltage pulses generated by the drive source 20 with the imaging controller 14, and assigns the X-ray image 11 with a suitable identifier according to an imaging position or irradiation position. The storage control unit 57 stores the X-ray image 11 with the identifier in a memory or storage medium. In FIG. 6, specifically, the storage control unit 57 assigns stop positions of the X-ray source 10 with respectively integer numbers of 1, 2, 3, . . . , N−1, N from the left end toward the right side in FIG. 1. The value N is a total number of events of imaging according to an imaging condition. The storage control unit 57 records the integer numbers in a file name, meta information and the like of the X-ray image 11 by way of identifiers, with which imaging positions of the image data can be recognized. Also, the total number N of events of imaging, and the angle θ=Θ/(N−1) of imaging positions of the X-ray images 11 adjacent with one another, are stored in association.

An angle Θ is defined (certain irradiation angle) between end lines (imaging positions for Nos. 1 and N of the X-ray images 11) of the entire moving area or initial search area (irradiation area) of the X-ray source 10 as viewed from the center of the receiving surface 18 of the imaging cassette 12. In relation to the angle Θ, an angle expressed by the left end of the entire moving area is zero (0), which means a reference angle. An angle between the left end of the moving area and the center position (half of the entire moving area) is Θ/2. An angle between the left end of the moving area and a point of one quarter of the entire moving area is Θ/4 or 3Θ/4. In the present description, the left end of the moving area is referred to a position 0. A right end of the moving area is referred to a position 1. A point determined by equal division of 2ⁿ⁻¹ for the moving area is referred to a position k/2ⁿ⁻¹. Note that n is an integer equal to or more than 2, and k is an integer equal to or more than 1. A position next to the left end of the moving area is k=1. A position next to the right end of the moving area is k=2ⁿ⁻¹−1. The value of k changes incrementally from the left end to the right end.

The search processing unit 58 for search support includes a searcher 58a and an acquisition device 58b (acquisition means), and when the interface control unit 55 responds to a command signal for search from the input unit 48, acquires the X-ray image 11 from the storage medium 42. The interface control unit 55 drives the display panel 47 to display the X-ray image 11 in the display window.

When images in a stereo image pair are displayed on the display window, at first the search processing unit 58 acquires two of the X-ray images 11 among such obtained in tomosynthesis imaging of one sequence, the two being obtained from positions approximately symmetrical with one another with respect to a center of the moving area of the X-ray source 10 (center of the support shaft). Note that the two images in a pair among the X-ray images 11 are herein referred to parallax images (stereo image pair) in relation to the stereoscopic imaging. Let a view center be a point of acquiring parallax images, for example, the center position of the moving area of the X-ray source 10.

For parallax images, acquisition is made according to the parallax angle Φ. The parallax angle Φ is an angle between positions of the two parallax images as viewed from the center of the receiving surface 18 of the imaging cassette 12. There are predetermined optimized values of the parallax angle Φ for depth of view in the stereoscopy. For example, let the parallax angle Φ be determined at an angle θ between two of the X-ray images 11 of which identifiers are next to one another. Let the total number N be an even number. Then two of the X-ray images 11 with the identifiers of N/2 and (N/2)+1 are selected as parallax images with respect to the center of the support shaft as indicated by the hatching in FIG. 6.

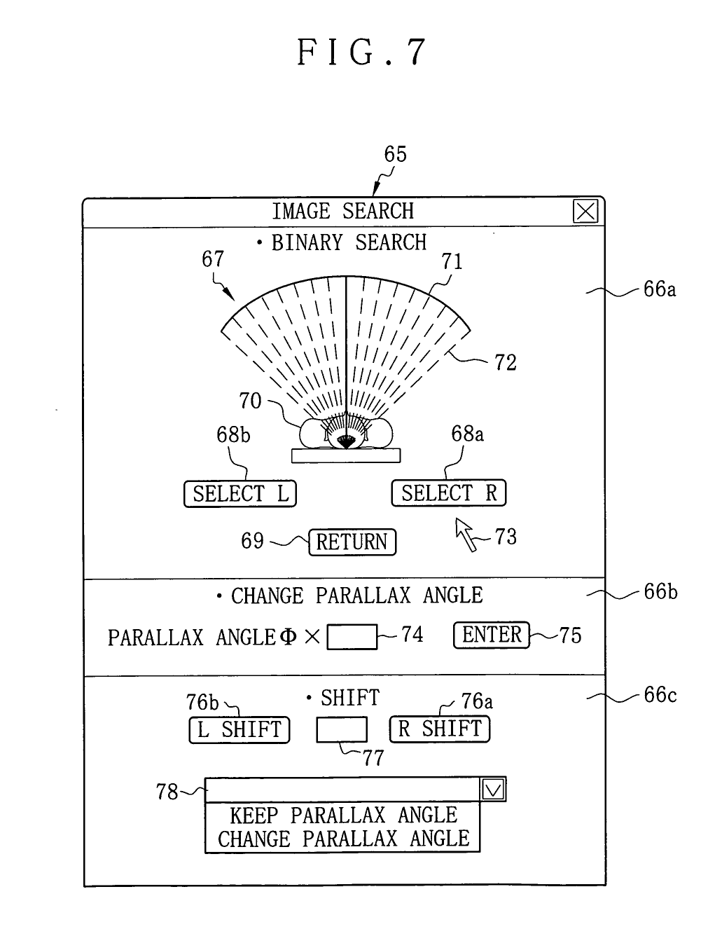

The input unit 48, when a radiologist wishes to display a 3D image on the display window, is operated manually. An image search window 65 or image selection window of FIG. 7 is caused by the interface control unit 55 to appear on the display panel 47 in response to the operation of the input unit 48.

In FIG. 7, the image search window 65 includes a first display region 66a, a second display region 66b and a third display region 66c. Various operation buttons are indicated in the display regions 66a-66c in a form of the GUI.

The first display region 66a is for searching parallax images with search support in a multi-stage manner of the binary search, and includes a status diagram 67, a right button 68a, a left button 68b and a return button 69.

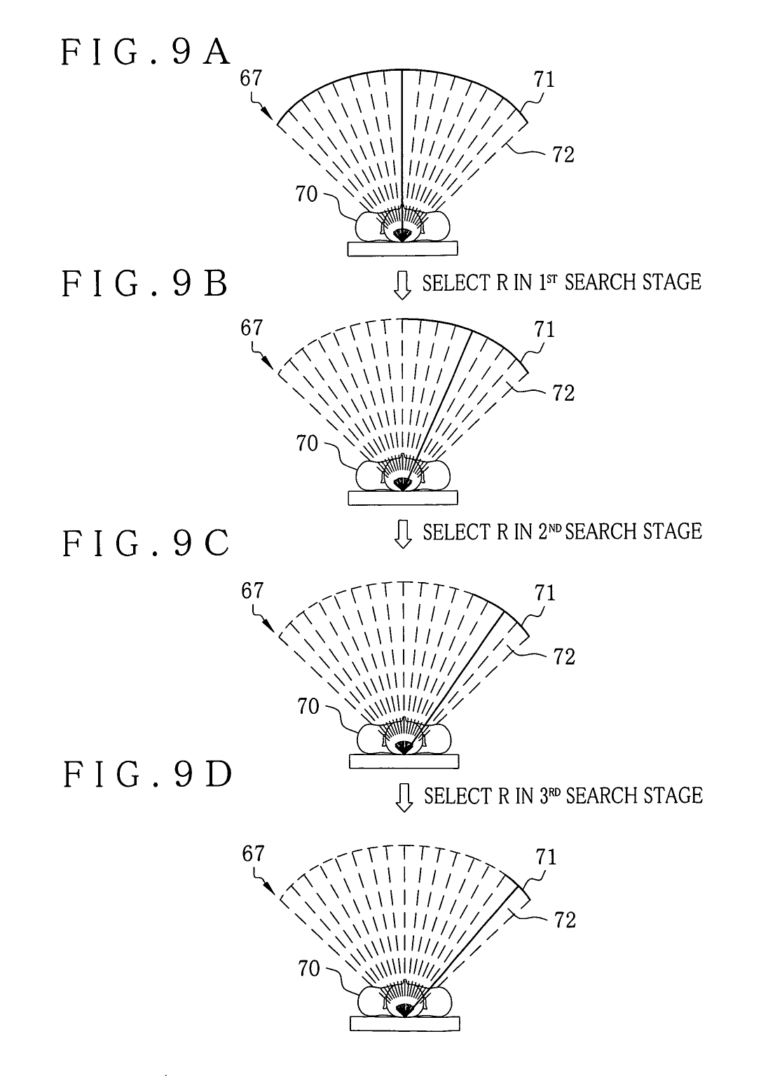

In the status diagram 67, a view center is illustrated as an imaging position of substantially parallax images displayed presently in the display window. A moving object image 70 or animation image is disposed on a lower side to indicate the cassette and the object P lying on the cassette. A moving area image 71 or animation image is disposed on an upper side to indicate a moving area of the X-ray source 10. A line segment 72 is drawn to extend between the moving object image 70 and the moving area image 71. The line segment 72 divides the moving area by a value of 2ⁿ. In the moving area image 71 of a moving area of the X-ray source 10, a selected portion by use of the right and left buttons 68a and 68b is indicated by the solid line, and a remaining unselected portion is indicated by the broken line. Among a plurality of line segments 72, the solid line indicates a view center of parallax images displayed presently in the display window. The broken line indicates the remainder of the line segments 72. The display of the solid and broken lines of the moving area image 71 and the line segments 72 (setting region) is changed over by use of the right and left buttons 68a and 68b. See FIG. 9.

In FIG. 7, a total number N of events of imaging is 40. The angle Θ is 160 degrees. The moving area image 71 is divided by 16 to form 17 line segments 72. A center one of the line segments 72 is drawn as a solid line. The moving area image 71 is drawn with solid lines. Parallax images obtained nearly symmetrically with reference to the center of the moving area of the X-ray source 10 are in a default status as depicted in the drawing.

The right button 68a as determining region is operated to specify a right area in FIG. 1 within a half area obtained by dividing the entire moving area or initial search area of the X-ray source 10. Also, the right button 68a is operated to specify a right area in FIG. 1 within a half area (½ⁿ of the entire moving area) obtained by dividing a previously specified moving area by use of the right and left buttons 68a and 68b. Details of the left button 68b are reverse to those of the right button 68a.

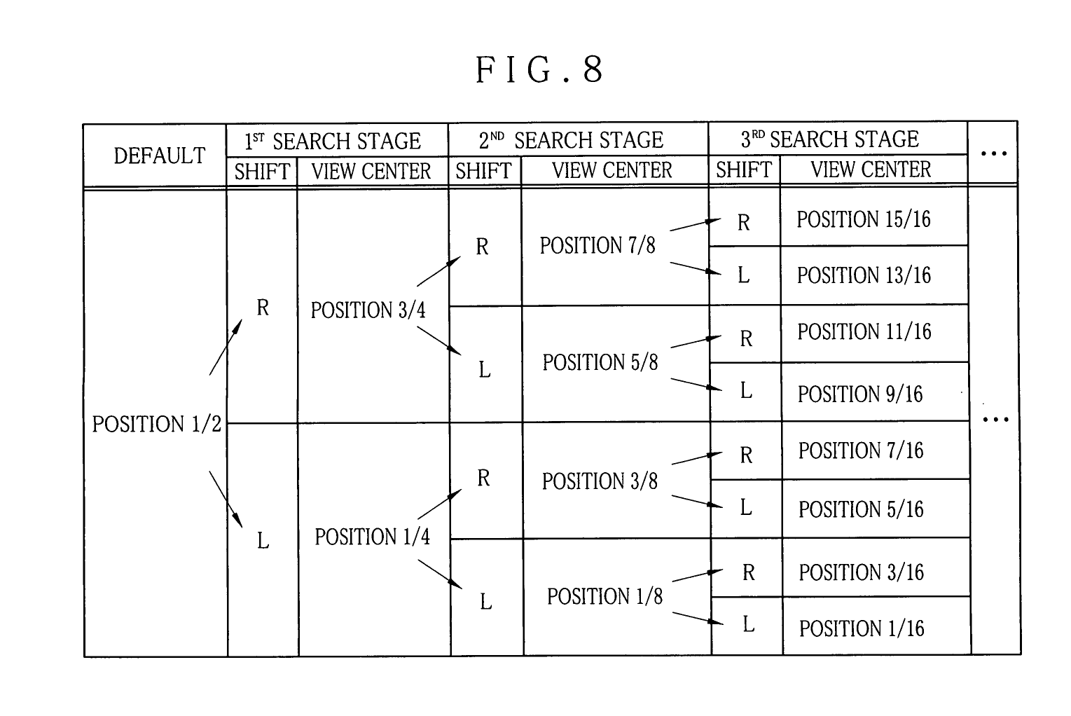

In FIG. 8, a pointer 73 is illustrated. In a default state before the preliminary search of a first search stage, parallax images are displayed as created in positions symmetric with one another with respect to a center position of the moving area of the X-ray source 10. Then the pointer 73 is set at one of the right and left buttons 68a and 68b and depressed with a click, as the first search stage of the preliminary search. Then the search processing unit 58 acquires two of the X-ray images 11 with the parallax angle Φ as parallax images from the storage medium 42 after creation in positions symmetric with one another with respect to a center position of the area of ½ selected by use of the right or left button 68a or 68b. Note that the center position is a position of ¾ (angle 3Θ/4) of a moving area of the X-ray source 10 if the right button 68a is pushed selectively, or is a position of ¼ (angle Θ/4) of the moving area of the X-ray source 10 if the left button 68b is pushed selectively. The center position is a new view center.

In a second search stage with the right and left buttons 68a and 68b, one of quarter areas of the entire moving area or initial search area within the half area selected in the first search stage is selected. The searcher 58a extracts parallax images obtained symmetrically with one another with respect to a center position of the selected area, namely one of positions of ⅞, ⅝, ⅜ and ⅛. In a third search stage, one of one eighth areas of the entire moving area within the quarter area selected in the second search stage is selected. The searcher 58a extracts parallax images obtained symmetrical with one another with respect to a center position of the selected area, namely one of positions of 15/16, 13/16, 11/16, 9/16, . . . , 1/16. Similarly, in an nth search stage, one of ½ⁿ areas of the entire moving area within the previously selected area selected in the (n−1)th search stage is selected with the right and left buttons 68a and 68b. It is possible to operate the right and left buttons 68a and 68b immediately before an angle of the respective areas of equal division by 2ⁿ becomes smaller than the parallax angle Φ.

In FIG. 8, a position of ¾ (or 6/8) is a center position for retrieving parallax images in the preliminary search of a first search stage. In a second search stage of the fine search after the first, a center position for retrieving parallax images is a position of ⅞ (or (6+1)/8) after pushing the right button 68a, or is a position of ⅝ (or (6−1)/8) after pushing the left button 68b. In general, let a position of k/2ⁿ⁺¹ be a center position for retrieving parallax images in an (n−1)th search stage. A center position for retrieving parallax images in an nth search stage is expressed as a position of (k+1)/2ⁿ⁺¹ or (k−1)/2ⁿ⁺¹. The position of (k+1)/2ⁿ⁺¹ is determined after pushing the right button 68a. The position of (k−1)/2ⁿ⁺¹ is determined after pushing the left button 68b.

Let N be 40 as a total number of events of imaging. The parallax angle Φ=Θ. Let the right button 68a be depressed for first, second and third times. Then a pattern on the display window is changed over from the default position to the positions of ¾, ⅞ and 15/16. Specifically, the state of the default position has a 3D image according to images of identifiers (20, 21) of symmetry with respect to a position of ½. The second state has a 3D image according to images of identifiers (30, 31) of symmetry with respect to a position of ¾. The third state has a 3D image according to images of identifiers (35, 36) of symmetry with respect to a position of ⅞. The fourth state has a 3D image according to images of identifiers (37, 38) of symmetry with respect to a position of 15/16. In FIGS. 9A-9D, changes in the moving area image 71 in the status diagram 67 are illustrated. In FIG. 9A, the solid line indicates the default moving area as a full area. In FIG. 9B, a right half of the moving area after the first search stage is indicated by the solid line. In FIG. 9C, a right quarter of the moving area after the second search stage is indicated by the solid line. In FIG. 9D, one eighth of the right side of the moving area after the third search stage is indicated by the solid line. The line segment 72 is changed over to set the solid line at a center in the state of FIG. 9A, set the solid line at a position of ¾ (13th line) in the state of FIG. 9B, set the solid line at a position of ⅞ (15th line) in the state of FIG. 9C, and set the solid line at a position of 15/16 (16th line) in the state of FIG. 9D.

If N=40 and Φ=θ, the X-ray image 11 with the parallax angle Φ and symmetric to the position of 15Θ/16 (=0.9375Θ) does not exist. However, the X-ray image 11 with the identifier of 37 of the position of 36Θ/39 (=0.9231Θ), and the X-ray image 11 with the identifier of 38 of the position of 37Θ/39 (=0.9487Θ) are images meeting the condition of the parallax images the most closely. Thus, those of the X-ray images 11 are extracted.

In FIG. 7, the return button 69 is pushed for returning one step before pushing the right and left buttons 68a and 68b. For example, if no 3D image as desired is obtained after pushing the right button 68a in the first search stage, then the return button 69 is depressed to return to the default state. The left button 68b is pushed newly.

The second display region 66b is used for changing the parallax angle Φ, and includes an input field 74 and an enter button 75. In the input field 74, a numerical value is input by use of a keyboard. When the enter button 75 is depressed after inputting the numerical value in the input field 74, the parallax angle Θ of a default value is multiplied by the input numerical value to determine a new parallax angle Θ. The search processing unit 58, if there is a change in the parallax angle Θ with the second display region 66b, searches images according to the new parallax angle Θ.

The third display region 66c is an acquiring region associated with the acquisition device 58b, and includes a right shift button 76a, a left shift button 76b, an input field 77 and a mode selection field 78 or pull down menu. A triangular sign is indicated beside the mode selection field 78, and clicked to indicate two modes in the mode selection field 78 such as “Keep parallax angle” and “Change parallax angle”. When the mode “Keep parallax angle” is selected in the mode selection field 78, a desired value is input in the input field 77, and one of the shift buttons 76a and 76b is pushed, then parallax images in the display window are shifted to the right or left by the value input in the input field 77 without change in the parallax angle Θ. For example, the X-ray image 11 with the identifiers (30, 31) is displayed as parallax images initially. A value 1 is input and the right shift button 76a is pushed selectively. Then the X-ray image 11 with the identifiers (31, 32) is selected and displayed. If a value 2 is input, then the X-ray image 11 with the identifiers (28, 29) or (32, 33) is selected and displayed.

When “Change parallax angle” is selected in the mode selection field 78 and a suitable value desired by a radiologist is input in the input field 77, the shift button 76a or 76b is selectively pushed. Then a first one of the parallax images displayed presently in the display window is shifted to the right or left stepwise at a certain step with reference to a second one of the parallax images having a smaller value of the identifier. In short, the parallax angle Θ is changed between the first parallax image and the second parallax image which is the reference image. For example, the X-ray image 11 with an identifier (30, 31) is displayed as parallax images. A value 1 is input and the right shift button 76a is selectively pushed. Then the X-ray image 11 with an identifier (30, 32) is extracted and displayed. If a value 2 is input, then the X-ray image 11 with an identifier (28, 30) or (30, 33) is displayed.

The operation of the embodiment is described now. At first, a radiologist or radiographic technician as a medical service provider at the X-ray imaging system 2 makes the object P lie in a predetermined position to which the X-ray source 10 and the imaging cassette 12 are opposed. He or she inputs information of imaging conditions with the input interface 15 of the imaging controller 14, and instructs a start of imaging. In response, the imaging controller 14 actuates the drive source 20 of the moving device 13, to shift the X-ray source 10 to plural predetermined positions of the support shaft. At each time that the X-ray source 10 reaches one of those positions in the control of the imaging controller 14, X-rays are applied to the object P by the X-ray tube 17 of the X-ray source 10, and are detected by the X-ray detection device 19 of the imaging cassette 12. The detected component of the X-rays in the X-ray detection device 19 is converted by the signal processor 29 to the X-ray image 11 of a digital form, which is transmitted by the imaging controller 14 to the viewer terminal apparatus 16.

The receiver 56 in the viewer terminal apparatus 16 receives the X-ray image 11 from the imaging controller 14. The storage control unit 57 assigns information of an identifier to the X-ray image 11, and then stores the X-ray image 11 in the storage medium 42. When the radiologist operates the image search window 65 for search, the search processing unit 58 acquires the X-ray image 11 from the storage medium 42. The interface control unit 55 causes the display panel 47 to display the X-ray image 11 as parallax images (stereo image pair). The radiologist can view the parallax images on the display panel 47 at the viewer terminal apparatus 16.

For the search with the first display region 66a in the image search window 65, the right or left button 68a or 68b is selectively pushed to select one of two areas which have been determined by split of a selected area in an (n−1)th search stage. The search processing unit 58 acquires two of the X-ray images 11 as parallax images with a parallax angle Θ, the two being obtained in positions symmetric with one another with respect to a position of k/2ⁿ. The radiologist observes parallax images on the display panel 47 after the search in the search processing unit 58, recognizes one of the right and left areas where a desired parallax image is present, and pushes one of the right and left buttons 68a and 68b according to the recognition. He or she removes a remaining one of the areas from a target of the search, and narrows a search area in the multi-stage process of ½, ¼, ⅛ and the like.

The radiologist accesses the image search window 65 and searches parallax images the most suitable for diagnosis. At first, the first display region 66a is used for preliminarily determining a range of the search (in the searcher 58a). Then he or she uses the third display region 66c to shift images, to determine a finally selected pair of parallax images (in the acquisition device 58b). Also, he or she changes the parallax angle by use of the second display region 66b if required.

He or she inputs a command signal of creating a reconstructed image with an enhanced ROI or region of interest by use of the input unit 48 if the region of interest is found after the stereoscopic diagnosis, such as a lesion of a disease. The CPU 40 of the viewer terminal apparatus 16 in FIG. 3 creates the reconstructed image. The interface control unit 55 drives the display panel 47 to display the reconstructed images in the display window.

As described above, a search area for parallax images is limited by consecutively selecting one of the two areas by use of the right or left button 68a or 68b. Time required for obtaining desired parallax images can be shortened remarkably.

The number of the X-ray images 11 in one set of the tomosynthesis imaging is as high as 40-80. If a radiologist wishes to select his or her required parallax images among those in a simple manner, extraordinarily long time is required due to a high number of possible pairs of images. In contrast with this, search of the parallax images is carried out rapidly according to multi-stage selection in the binary search. Total time for the search can be shortened by the search support. Thus, the radiologist can work for diagnosis efficiently.

Also, it is possible to change a depth of a stereoscopic view of a stereo image pair according to preference of the radiologist, because the parallax angle can be changed. Stereo image pairs of the parallax images can be selectively determined by the search, so that parallax images can be retrieved according to fine consideration of the radiologist, because of the two search stages of the preliminary search with the first display region 66a and fine search.

In the above embodiment, the X-ray image 11 from the imaging controller 14 is stored in the storage medium 42 of the viewer terminal apparatus 16. Furthermore, a storage medium separate from the viewer terminal apparatus 16 can be used to store the X-ray image 11, such as an image database server and the like. The server for this purpose has a storage control unit and search processing unit, and receives a command signal from the viewer terminal apparatus 16 as a client, so that the search processing unit carries out acquisition of parallax images.

In the above embodiment, the X-ray imaging system 2 is an installed type in an X-ray room or examination room of a hospital. However, the X-ray imaging system 2 of the invention can be a portable type capable of X-ray imaging in an emergency site of an accident, disaster and the like or a home where a patient lives and is treated by a doctor, the portable type including the X-ray source 10, the imaging cassette 12, the moving device 13 and the imaging controller 14. In combination with this, an example of the input interface 15 is a personal computer, in which the application program 46 is installed in the same manner as the viewer terminal apparatus 16.

In the emergency medicine, patients require urgent transportation and treatment. Should time for selecting parallax images be very long, a patient' s life may be in peril. However, it is possible to select parallax images rapidly when the feature of the invention is used in the viewer terminal apparatus 16 of a portable type. Effects of the invention are obtained specifically in the emergency medicine.

In the embodiment, displayed parallax images before the first search stage of the preliminary search with the right and left buttons 68a and 68b are those obtained in positions symmetric with one another with respect to the center position of the moving area of the X-ray source 10. However, the present invention is not limited to this feature. Initially displayed parallax images may be those obtained in positions determined by a radiologist's preference and irrespective of the center position of the moving area of the X-ray source 10. In operation, he or she narrows a search area of parallax images by use of the right and left buttons 68a and 68b in the manner similar to the above embodiment. Typically when he or she estimates a preliminary large area of presence of parallax images according to experience or the like, preliminary search can be specifically suitable before fine search, so as to make the search processing more efficient for imaging.

Note that an area to be selected by use of the right and left buttons 68a and 68b is not ½ⁿ of the entire moving area or initial search area. This is a difference from the above embodiment. For example, a position of ⅕ is selected (angle Φ/5). The left button 68b is selectively pushed at a first search stage. Then a search area selected by the right and left buttons 68a and 68b is a 1/10 area of he entire moving area. If the right button 68a is selectively pushed, a selected search area is a ⅖ area.

If the earlier view center determined in a preceding search stage exists in a direction selected by the right or left button 68a or 68b, then a search area for parallax images is limited to an area defined between the presently determined view center and the most recent view center prior to the presently determined view center. If the earlier view center determined in a preceding search stage does not exist in a direction selected by the right or left button 68a or 68b, then a search area for parallax images is limited to an area defined between the presently determined view center and an irradiation end position as viewed in the selected direction. After the limitation, a view center of the limited search area is determined as a new view center. For example, according to the above embodiment, the right direction is selected for the first search stage and the left direction is selected for the second search stage. Namely, a change in the direction occurs. Then a limited search area is from the position of ¾ to the position of ½ the nearest to the position of ¾. A new view center is a position of ⅝ as a view center of the limited search area. In another example, the right direction is selected for the first and second search stages. Namely, no change in the direction occurs. Then a limited search area is from the position of ¾ to the position of 1. A new view center is a position of ⅞ as a view center of the limited search area.

The radiation imaging system of the invention is not limited to the above construction. Various modifications of the structure are possible in the scope of the invention.

In the above embodiment, the imaging controller 14 controls the X-ray detection device 19 for operation. However, the X-ray detection device 19 can operate without being controlled by the imaging controller 14. The X-ray detection device 19 can detect its emission of X-rays, and can operate according to the detection.

In the above embodiment, the X-ray detection device 19 is the direct conversion type. However, the X-ray detection device 19 may be an indirect conversion type, in which an incident component of X-rays is converted into visible light by a scintillator, and the visible light is converted into an electric signal by use of a solid state detector such as amorphous silicon a-Si and the like.

In the above embodiments, the imaging cassette 12 and the moving device 13 are connected to the imaging controller 14 in a wired manner. However, the imaging cassette 12 and the moving device 13 can be connected wirelessly to the imaging controller 14. For this structure, a battery as a power source is incorporated in each of the imaging cassette 12 and the moving device 13.

Also, any one of the imaging cassette control unit 27, the motion control unit 30 and the drivers 28 and 31 maybe associated with the imaging cassette 12 or the moving device 13 instead of the imaging controller 14. The high voltage source 26 may be a separate component and can be externally connected to the imaging controller 14.

Also, it is possible to create a reconstructed image in the imaging controller 14 instead of the viewer terminal apparatus 16. The X-ray source 10 can be moved manually without use of the drive source 20. Also, a moving mechanism can be added to the imaging cassette 12, and can move the imaging cassette 12 in synchronism in a direction opposite to moving of the X-ray source 10.

It is also possible to construct a support shaft or moving shaft of a moving device extendable vertically so as to adjust a moving area and adjust an SID or source image distance from the X-ray source 10 to the receiving surface 18.

In general, the maximum angle of projection of the X-ray source 10 through a collimator opening is as large as 12 degrees. If a radiologist wishes to change the field of irradiation of X-rays without changing the size of the collimator opening, then the support shaft or moving shaft of the moving device is moved up or down to adjust the SID. Note that the maximum angle of projection is a vertex angle of an isosceles triangle which is formed with a base along a line between two distal ends of the collimator opening and with a vertex at the focal point of the X-ray tube 17.

The radiation in the radiation imaging system of the present invention, although X-rays according to the above embodiment, can be other radiation such as gamma rays and the like. Also, the radiation can be visible light. The present invention can be used in image search apparatus and method, and a user interface for image search, having various structures.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. An image processing apparatus for plural images obtained by imaging of an object upon application of radiation at plural irradiation angles from irradiation positions within an area between first and second irradiation positions, said image processing apparatus comprising:
a display control unit for displaying two of said images on a display panel stereoscopically with a predetermined parallax angle, said two images being formed with said radiation from irradiation positions which are symmetric with one another with respect to a view center determined specifically within said area between said first and second irradiation positions;
a first input unit for selecting a shift direction of said view center from forward and backward directions defined between said first and second irradiation positions;
a search processing unit for checking whether an earlier view center specified previously is located on a specified side extending in said shift direction from said view center being current, and if said earlier view center is located on said specified side, determining a search area between said current view center and a selected one of earlier view centers which is nearest to said current view center, and if said earlier view center is located opposite to said specified side, determining a search area between said current view center and a position which is located on said specified side and farthest from said current view center;
an acquisition device adapted to acquiring two new images from said search area for display on said display panel by determining a new view center thereof at a center of said search area.

2. An image processing apparatus as defined in claim 1, wherein initially before operating said first input unit, said view center is a center between said first and second irradiation positions.

3. An image processing apparatus as defined in claim 1, further comprising a second input unit for changing said parallax angle.

4. An image processing apparatus as defined in claim 1, further comprising a second input unit for shifting said view center to change over said two images on said display panel to two other images.

5. An image processing apparatus as defined in claim 1, further comprising a second input unit for changing said parallax angle with reference to a first image of said two images in order to change over a second image of said two images displayed on said display panel to another image.

6. An image processing apparatus as defined in claim 1, further comprising an image processing device for creating a tomographic image according to said two images by adding up image data of said plural images and by enhancing a region of interest of said object.

7. An image processing apparatus as defined in claim 1, wherein said image processing apparatus is a computer apparatus adapted to viewing said two images stereoscopically and editing a medical report.

8. An image processing apparatus as defined in claim 1, wherein said image processing apparatus is a computer apparatus connected to a radiation imaging system having an increased system portability, and adapted to viewing said two images stereoscopically.

9. A radiation imaging system having a radiation imaging apparatus and an image processing apparatus, comprising:
said radiation imaging apparatus including:
a radiation source for applying radiation to an object;
a detection device for image forming by detecting said radiation transmitted through said object;
a moving device for moving said radiation source to direct said radiation at plural irradiation angles from irradiation positions within an area between first and second irradiation positions;
said image processing apparatus including:
a display control unit for displaying two images on a display panel stereoscopically with a predetermined parallax angle, said two images being formed with said radiation from irradiation positions which are symmetric with one another with respect to a view center determined specifically within said area between said first and second irradiation positions;
a first input unit for selecting a shift direction of said view center from forward and backward directions defined between said first and second irradiation positions;
a search processing unit for checking whether an earlier view center specified previously is located on a specified side extending in said shift direction from said view center being current, and if said earlier view center is located on said specified side, determining a search area between said current view center and a selected one of earlier view centers which is nearest to said current view center, and if said earlier view center is located opposite to said specified side, determining a search area between said current view center and a position which is located on said specified side and farthest from said current view center;
an acquisition device adapted to acquiring two new images from said search area for display on said display panel by determining a new view center thereof at a center of said search area.

10. A radiation imaging system as defined in claim 9, wherein said moving device arcuately moves said radiation source.

11. An image processing method for plural images obtained by imaging of an object upon application of radiation at plural irradiation angles from irradiation positions within an area between first and second irradiation positions, said image processing method comprising steps of:
searching two images formed with said radiation from irradiation positions which are symmetric with one another with respect to a view center determined specifically within said area between said first and second irradiation positions;
displaying said two images on a display panel stereoscopically with a predetermined parallax angle;
selecting a shift direction of said view center from forward and backward directions defined between said first and second irradiation positions, in order to change over said two images on said display panel;
checking whether an earlier view center specified previously is located on a specified side extending in said shift direction from said view center being current;
if said earlier view center is located on said specified side, determining a search area between said current view center and a selected one of earlier view centers which is nearest to said current view center;
if said earlier view center is located opposite to said specified side, determining a search area between said current view center and a position which is located on said specified side and farthest from said current view center;
acquiring two new images from said search area for display on said display panel by determining a new view center thereof at a center of said search area;
displaying said two new images on said display panel stereoscopically.

12. A computer executable program for image processing of plural images obtained by imaging of an object upon application of radiation at plural irradiation angles from irradiation positions within an area between first and second irradiation positions, said computer executable program comprising:
a displaying program code for displaying two of said images on a display panel stereoscopically with a predetermined parallax angle, said two images being formed with said radiation from irradiation positions which are symmetric with one another with respect to a view center determined specifically within said area between said first and second irradiation positions;
a selecting program code for selecting a shift direction of said view center from forward and backward directions defined between said first and second irradiation positions;
a processing program code for checking whether an earlier view center specified previously is located on a specified side extending in said shift direction from said view center being current, and if said earlier view center is located on said specified side, determining a search area between said current view center and a selected one of earlier view centers which is nearest to said current view center, and if said earlier view center is located opposite to said specified side, determining a search area between said current view center and a position which is located on said specified side and farthest from said current view center;
an acquiring program code for acquiring two new images from said search area for display on said display panel by determining a new view center thereof at a center of said search area.
